(19) United States
(12) Patent Application Publication
(10) Pub. No.: US 2010/0286479 A1
(43) Pub. Date: Nov. 11, 2010

(54) INTERNAL PRESSURE DETECTING DEVICE FOR INFLATING AND DEFLATING MEMBER AND ENDOSCOPE APPARATUS

(76) Inventors: Tsuyoshi ASHIDA, Ashigarakami-gun (JP); Shinichi YAMAKAWA, Ashigarakami-gun (JP); Takayuki NAKAMURA, Ashigarakami-gun (JP)

Correspondence Address: BIRCH STEWART KOLASCH & BIRCH, PO BOX 747, FALLS CHURCH, VA, 22040-0747, US

(21) Appl. No.: 12/777,093

(22) Filed: May 10, 2010

(30) Foreign Application Priority Data
May 11, 2009 (JP) 2009-114768

(51) Int. Cl. A61B 1/00; B65B 3/26

(52) U.S. Cl. 600/116; 141/197; 600/207

(57) ABSTRACT

An internal pressure detecting device can accurately detect an internal pressure of the inflating and deflating member such as a balloon which is provided at an intracavital insertion tool such as an endoscope, an endoscope auxiliary tool or an endoscope treatment tool, is provided. The internal pressure detecting device has: a pressure detection conduit line which is provided separately from a supply and exhaust conduit line through which a fluid is supplied to or exhausted from an inside of an inflating and deflating member provided at an intracavital insertion tool, and which communicates with the inside of the inflating and deflating member; and a pressure detecting device which is connected to the pressure detection conduit line and detects an internal pressure of the inflating and deflating member.

### CROSS-REFERENCE TO RELATED APPLICATIONS

This application claims the priority benefit under 35 U.S.C. §119 of Japanese Patent Application No. 2009-114768 filed on May 11, 2009, which is hereby incorporated in its entirety by reference.

### BACKGROUND OF THE INVENTION

1. Field of the Invention

The presently disclosed subject matter relates to an internal pressure detecting device for an inflating and deflating member and an endoscope apparatus, and particularly relates to an internal pressure detecting device for an inflating and deflating member provided in an intracavital insertion tool such as an endoscope, an endoscope auxiliary tool or an endoscope treatment tool, and an endoscope apparatus having the device.

2. Description of the Related Art

Japanese Patent Application Laid-Open No. 2002-301019 discloses the art for detecting the internal pressure of a balloon by attaching a pressure sensor in the vicinity of a pump via a supply and exhaust conduit line for supplying and exhausting the gas to and from the balloon which is provided at a distal end portion of an endoscope or an endoscope auxiliary tool.

### SUMMARY OF THE INVENTION

However, in the prior art of Japanese Patent Application Laid-Open No. 2002-301019, a pressure loss in the supply and exhaust conduit line occurs in accordance with gas supply and exhaust flow velocity during supply and exhaust of the gas, and accurate internal pressure in the balloon cannot be detected. More specifically, when the gas is supplied to the balloon, the pressure value measured by the pressure sensor becomes the value which is the result of adding the pressure loss to the real internal pressure of the balloon. That is, “the pressure at the pressure sensor position=the balloon internal pressure+the pressure loss of the supply and exhaust conduit line” is obtained. Further, when the gas is exhausted from the balloon, the pressure value measured by the pressure sensor becomes the value which is the result of subtracting the pressure loss from the real internal pressure of the balloon. That is, “the pressure at the pressure sensor position=the balloon internal pressure−the pressure loss of the supply and exhaust conduit line” is obtained.

Therefore, in increasing the internal pressure of the balloon to a predetermined pressure P0, even when the internal pressure of the balloon does not actually reach the predetermined pressure P0, the pump is controlled so that “the balloon internal pressure+the pressure loss of the supply and exhaust conduit line” becomes the predetermined pressure P0. Accordingly, the pump is suppressedly operated, and its maximum capacity is not efficiently used, and much time is required for inflating the balloon as a result. Further, when the fluid is exhausted from the balloon, the pump is suppressedly operated under the influence of the pressure loss of the supply and exhaust conduit line, similar to the case of supply, and much time is required for deflating the balloon as a result.

The presently disclosed subject matter is made in view of the above circumstances, and has an object to provide an internal pressure detecting device for an inflating and deflating member and an endoscope apparatus which can accurately detect an internal pressure of the inflating and deflating member such as a balloon which is provided at an intracavital insertion tool such as an endoscope, an endoscope auxiliary tool, or an endoscope treatment tool.

In order to attain the above-described object, an internal pressure detecting device for an inflating and deflating member according to an aspect, includes: a pressure detection conduit line which is provided separately from a supply and exhaust conduit line through which a fluid is supplied to or exhausted from an inside of an inflating and deflating member provided at an intracavital insertion tool, and which communicates with the inside of the inflating and deflating member; and a pressure detecting device which is connected to the pressure detection conduit line and detects an internal pressure of the inflating and deflating member.

According to the aspect of the presently disclosed subject matter, since the pressure detection conduit line which is provided separately from the supply and exhaust conduit line, and communicates with the inside of the inflating and deflating member, and a pressure detecting device which is connected to the pressure detection conduit line and detects an internal pressure of the inflating and deflating member are provided, the internal pressure of the inflating and deflating member provided at the intracavital insertion tool can be accurately detected.

In one mode of the presently disclosed subject matter, an opening of the pressure detection conduit line is included in the inside of the inflating and deflating member.

According to this mode, the internal pressure of the inflating and deflating member provided at the intracavital insertion tool can be accurately detected without being influenced by the pressure loss of the supply and exhaust conduit line.

In one mode of the presently disclosed subject matter, an opening of the pressure detection conduit line is included in a vicinity of the inflating and deflating member in the supply and exhaust conduit line.

According to this mode, the influence of the pressure loss of the supply and exhaust conduit line is reduced, and the internal pressure of the inflating and deflating member provided at the intracavital insertion tool can be accurately detected.

In one mode of the presently disclosed subject matter, the pressure detecting device is disposed outside the intracavital insertion tool.

According to this mode, the intracavital insertion tool can be made compact.

In one mode of the presently disclosed subject matter, the pressure detection conduit line is provided inside the intracavital insertion tool.

According to this mode, the intracavital insertion tool can be made compact.

In one mode of the presently disclosed subject matter, the intracavital insertion tool is an insertion part of an endoscope.

According to this mode, the internal pressure of the inflating and deflating member provided at the insertion part of the endoscope can be accurately detected.

In one mode of the presently disclosed subject matter, the intracavital insertion tool is an endoscope auxiliary tool through which an insertion part of an endoscope is inserted.

According to this mode, the internal pressure of the inflating and deflating member provided at the endoscope auxiliary tool can be accurately detected.

In one mode of the presently disclosed subject matter, the intracavital insertion tool is an endoscope treatment tool which is led out from a forceps port of an endoscope.

According to such a mode, the internal pressure of the inflating and deflating member provided at the endoscope treatment tool can be accurately detected.

In one mode of the presently disclosed subject matter, the intracavital insertion tool is an insertion part of an endoscope, and the insertion part includes, in sequence from a proximal end side: a flexible part having flexibility; a bending part which is connected to a proximal end side of the flexible part and is bendable; and a distal end part which can be directed to a desired direction by bending the bending part, and the vicinity of the inflating and deflating member is a vicinity of a part where the bending part is connected to the flexible part.

According to this mode, the influence of the pressure loss in the supply and exhaust conduit line is reduced, and the internal pressure of the inflating and deflating member provided at the insertion part of the endoscope can be accurately detected.

In order to attain the above described object, an endoscope apparatus of the presently disclosed subject matter according to an aspect, includes: an intracavital insertion tool which is inserted into a body cavity; an inflating and deflating member provided at the intracavital insertion tool; a supply and exhaust conduit line through which a fluid is supplied to or exhausted from an inside of the inflating and deflating member; a supply and exhaust device which is connected to the supply and exhaust conduit line and supplies or exhausts the fluid to or from the inside of the inflating and deflating member; a pressure detection conduit line which is provided separately from the supply and exhaust conduit line and communicates with the inside of the inflating and deflating member; a pressure detecting device which is connected to the pressure detection conduit line and detects an internal pressure of the inflating and deflating member; and an internal pressure control section which controls the internal pressure of the inflating and deflating member by controlling the supply and exhaust device based on a detection result of the internal pressure detecting device.

According to the presently disclosed subject matter, until the internal pressure of the inflating and deflating member provided at the intracavital insertion tool reaches a predetermined pressure, the supply and exhaust device can be controlled to maximize its capacity when the fluid is supplied into the inflating and deflating member, or is exhausted from the inflating and deflating member. Accordingly, safe and quick supply and exhaust of the fluid are enabled.

According to the presently disclosed subject matter, the internal pressure of the inflating and deflating member such as a balloon which is provided at the intracavital insertion tool such as an endoscope, an endoscope auxiliary tool or an endoscope treatment tool can be accurately detected.

### BRIEF DESCRIPTION OF THE DRAWINGS

FIG. 1 is a system configuration view showing one example of an endoscope apparatus of a first embodiment;

FIG. 2 is an enlarged perspective view of a distal end part;

FIG. 3 is a sectional view of an area around a distal end of an insertion part;

FIG. 4 is a schematic configuration diagram of an inside of a balloon control device;

FIG. 5 is a sectional view of an area around a distal end of an insertion part showing a modified example of the first embodiment;

FIG. 6 is a system configuration view showing one example of an endoscope apparatus of a second embodiment;

FIG. 7 is a plane view showing an appearance of an insertion auxiliary tool;

FIGS. 8A and 8B are sectional views of a distal end part of a tube main body;

FIG. 9 is a system configuration view showing one example of an endoscope apparatus of a third embodiment; and

FIG. 10 is a sectional view of a side surface of an insertion guide tool.

### DETAILED DESCRIPTION OF THE PREFERRED EMBODIMENTS

Hereinafter, preferred embodiments of the presently disclosed subject matter will be described in detail in accordance with the attached drawings.

### First Embodiment

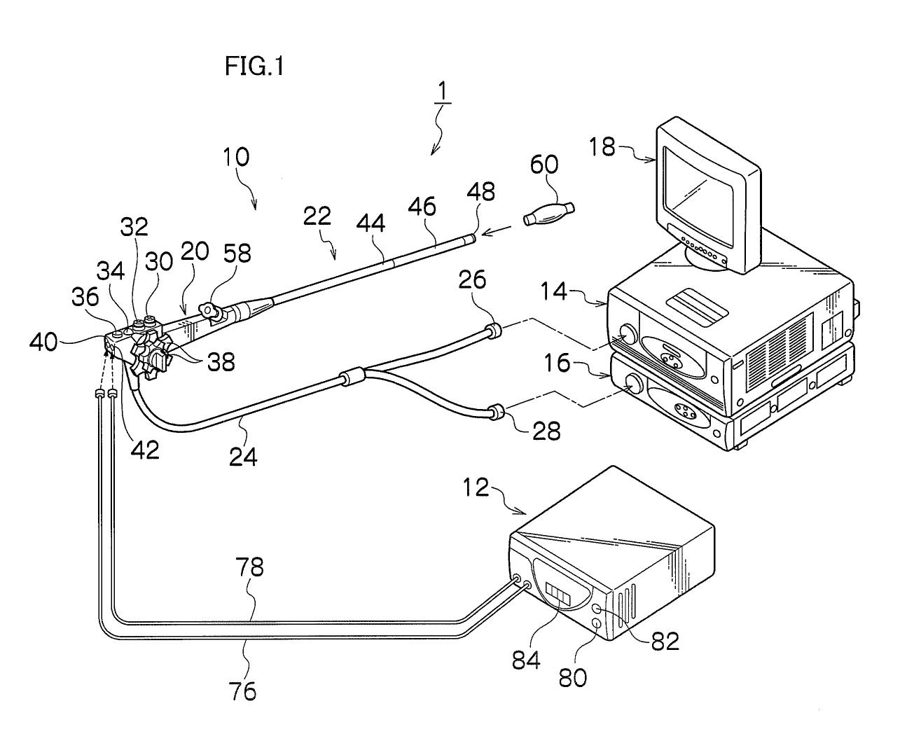

FIG. 1 is a system configuration view showing one example of an endoscope apparatus 1 of first embodiment as an endoscope apparatus having an internal pressure detecting device for an inflating and deflating member according to the presently disclosed subject matter.

As shown in FIG. 1, the endoscope apparatus 1 includes an endoscope 10, a balloon control device 12, a light source device 14, a processor 16 and a monitor 18.

The endoscope 10 includes a hand operation part 20 and an insertion part 22 which is configured to connect to the hand operation part 20 and is inserted into a body cavity. A universal cable 24 is connected to the hand operation part 20, and a light source connector 26 and an electrical connector 28 are provided at a distal end of the universal cable 24. The light source connector 26 is attachably and detachably connected to the light source device 14, and thereby, an illumination light is sent to an illumination optical system (not illustrated) provided at a distal end of the insertion part 22. Further, the electrical connector 28 is attachably and detachably connected to the processor 16.

On the hand operation part 20, an air supply/water supply button 30, a suction button 32, a shutter button 34 and a function switching button 36 are provided side by side, and a pair of angle knobs 38 and 38 are provided. A supply and exhaust conduit line port 40 is formed at a proximal end portion of the hand operation part 20. A fluid is supplied into the supply and exhaust conduit line port 40, or is exhausted from the supply and exhaust conduit line port 40, and thereby, a balloon 60 which will be described later can be inflated or deflated. As a fluid to be used, air, inert gas, water or the like can be properly selected.

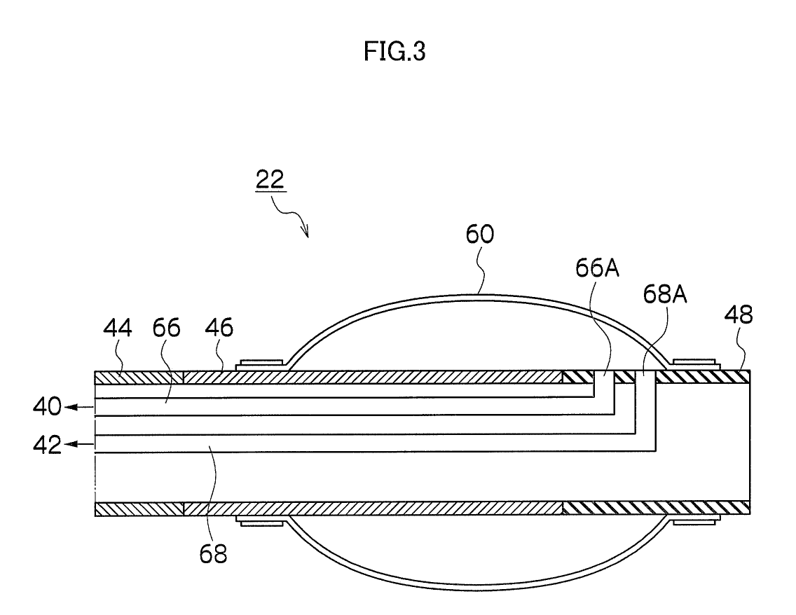

Further, a pressure detection conduit line port 42 is further formed at the proximal end portion of the hand operation part 20. As will be described later, a pressure detection conduit line 68 (see FIG. 3) which extends from the pressure detection conduit line port 42 communicates with the balloon 60.

The insertion part 22 is configured by a flexible part 44, a bending part 46 and a distal end part 48 in sequence from the hand operation part 20 side. The flexible part 44 is a part having sufficient flexibility, and is connected to a proximal end side of the bending part 46.

The bending part 46 is configured to bend remotely by rotating the angle knobs 38 and 38 of the hand operation part 20. For example, in the bending part 46, a plurality of cylindrical nodal rings are rotatably connected with guide pins, and a plurality of operation wires are inserted through the inside of the nodal rings to cause the guide pins to guide the operation wires. By performing pushing and pulling operations of the operation wires, the nodal rings are rotated so that the bending operation of the bending part 46 is performed. By performing a bending operation of the bending part 46, the distal end part 48 can be directed to a desired direction.

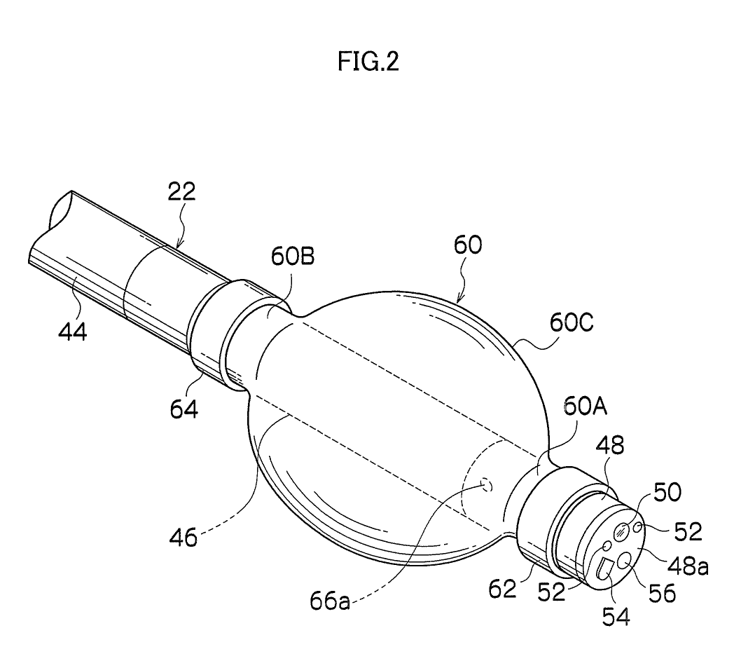

FIG. 2 is an enlarged perspective view of the distal end part 48. As shown in FIG. 2, a distal end surface 48a of the distal end part 48 is provided with an observation optical system 50, illumination optical systems 52 and 52, an air supply/water supply nozzle 54 and a forceps port 56.

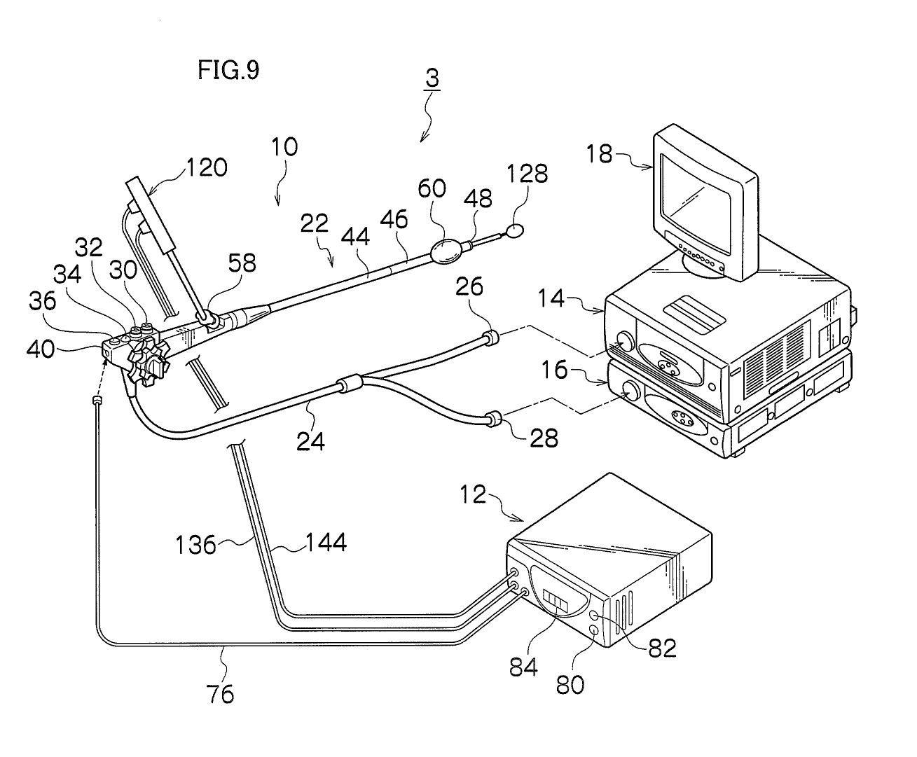

The forceps port 56 communicates with a forceps insertion part 58 shown in FIG. 1. Therefore, by inserting treatment tools such as forceps and an endoscope insertion guide tool from the forceps insertion part 58, the treatment tools can be led out from the forceps port 56. As the details will be described later, the endoscope insertion guide tool 120 (see FIG. 9) is a treatment tool which includes a balloon 128, and is for guiding insertion of the insertion part 22 of the endoscope 10.

Incidentally, as shown in FIG. 2, the balloon 60 formed by an elastic body such as rubber is fitted on an outer periphery surface of the insertion part 22. The balloon 60 is formed into a substantially cylindrical shape constricted at its end portions. The balloon 60 includes distal end part 60A and proximal end part 60B each of which has a small diameter, and a central swelling part 60C. The balloon 60 is fixed to the insertion part 22 by having the insertion part 22 inserted through the balloon 60 and being disposed at a predetermined position of the insertion part 22, and thereafter, fitting rubber rings 62 and 64 onto the distal end part 60A and the proximal end part 60B. As shown in FIG. 2, the balloon 60 is disposed over the area from the distal end part 48 to the bending part 46 in this case.

The method for fixing the distal end part 60A and the proximal end part 60B is not especially limited, and they may be fixed by winding a thread around them. Further, the shape of the balloon 60 is not limited to the substantially cylindrical shape constricted at the end portions, but may be formed into a spherical shape or the like.

FIG. 3 is a sectional view of an area around the distal end of the insertion part 22. As shown in FIG. 3, a supply and exhaust conduit line 66 which communicates with the supply and exhaust conduit line port 40 (see FIG. 1) at its proximal end side and which supplies and exhausts a fluid in the balloon 60, is formed inside the insertion part 22. The distal end side of the supply and exhaust conduit line 66 is configured to communicate with the balloon 60 via an opening 66A which is formed in the outer peripheral surface of the distal end part 48 and is included in the inside of the balloon 60.

Further, a pressure detection conduit line 68 the proximal end side of which communicates with the aforementioned pressure detection conduit line port 42 (see FIG. 1) is formed inside the insertion part 22. The distal end side of the pressure detection conduit line 68 is caused to communicate with the balloon 60 through an opening 68A which is formed in the outer peripheral surface of the distal end part 48 and is included in the inside of the balloon 60.

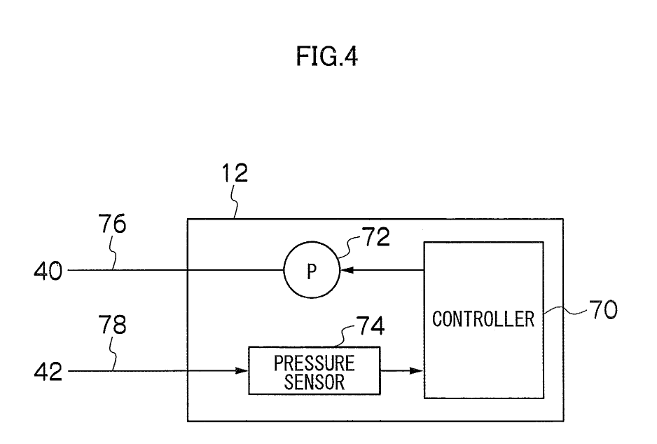

FIG. 4 is a schematic configuration diagram of the inside of the balloon control device 12. As shown in FIG. 4, the balloon control device 12, which is disposed outside the insertion part 22, includes a controller 70, a pump 72, a pressure sensor 74 and the like are configured inside the balloon control device 12. The pump 72 is connected to the aforementioned supply and exhaust conduit line port 40 (see FIG. 1) through a tube 76. Therefore, the pump 72 is controlled by the controller 70 to supply or exhaust the fluid, whereby the balloon 60 can be inflated or deflated through the tube 76, the supply and exhaust conduit line port 40 and the supply and exhaust conduit line 66 (see FIG. 3). The balloon 60 is inflated into a substantially spherical shape by supplying the fluid, and sticks onto the outer surface of the insertion part 22 by exhausting the fluid.

Further, the pressure sensor 74 is connected to the pressure detection conduit line port 42 (see FIG. 1) through a tube 78. Therefore, the internal pressure of the balloon 60 can be detected by the pressure sensor 74 through the pressure detection conduit line port 42 and the tube 78.

Returning to FIG. 1, a power supply switch 80, a stop switch 82, a pressure display section 84 and the like are provided on the front surface of the balloon control device 12. The pressure display section 84 is a panel for displaying the pressure value of the balloon 60, and an error cord is displayed on the pressure display section 84 at the time of occurrence of abnormality such as a balloon break or the like.

The balloon control device 12 which is configured as described above controls the pump 72 with the controller 70, and supplies the fluid to the balloon 60 to inflate the balloon. Subsequently, the balloon control device 12 detects the internal pressure of the balloon 60 with the pressure sensor 74, and controls the pump 72 with the controller 70 based on the detection result to control the internal pressure of the balloon 60 to a constant value. Further, the balloon control device 12 controls the pump 72 with the controller 70 to exhaust the fluid from the balloon 60 to deflate the balloon, and similarly controls the internal pressure of the balloon 60 to a constant value.

The balloon control device 12 may be provided with a monitor (not illustrated) dedicated for a balloon which displays the internal pressure value, and inflated or deflated state of the balloon 60 when inflating or deflating the balloon 60. Further, the internal pressure value, and the inflated or the deflated state of the balloon 60 may be superimposed on the observation image of the endoscope 10 to be displayed on the monitor 18.

As one example of the operation method of the endoscope apparatus 1 which is configured as described above, the insertion part 22 is inserted by a push method, and the balloon 60 is inflated in accordance with necessity to fix the insertion part 22 to the inside of a body (for example, a large intestine). Subsequently, the insertion part 22 is pulled so that the canal shape of the inside of the body (for example, a large intestine) is simplified. After that, the balloon 60 is deflated, and the insertion part 22 is further inserted into the deep part of the enteric canal.

For example, the insertion part 22 is inserted from the anus of a subject, and when the distal end of the insertion part 22 passes the colon sigmoideum, the balloon 60 is inflated to fix the insertion part 22 to the enteric canal. Next, the insertion part 22 is pulled to make the colon sigmoideum substantially rectilinear. Next, the balloon 60 is deflated, and the distal end of the insertion part 22 is inserted into the deep part of the enteric canal. Thereby, the insertion part 22 can be inserted into the deep part of the enteric canal.

When the balloon 60 is to be inflated or deflated, the internal pressure of the balloon 60 is detected by the pressure sensor 74, and the pump 72 is controlled.

Here, in the present embodiment, the pressure detection conduit line 68 is provided separately from the supply and exhaust conduit line 66 as shown in FIG. 3. The internal pressure of the balloon 60 is detected by the pressure sensor 74 via the pressure detection conduit line 68, the pressure detection conduit line port 42 and the tube 78 (see FIG. 1).

Therefore, detection is not influenced by the pressure loss of the fluid which occurs in the supply and exhaust conduit line 66, and the fluid does not flow into the pressure detection conduit line 68 so that a pressure loss does not occur. Therefore, the internal pressure of the balloon 60 can be accurately detected by the pressure sensor 74 without depending on the supply and exhaust amount of the fluid to and from the balloon 60.

Accordingly, until the internal pressure of the balloon 60 reaches a predetermined pressure, the pump 72 can be controlled to be driven with the maximum capacity so that the fluid is supplied into the balloon 60 or is exhaust from the inside of the balloon 60, and safe and quick supply and exhaust are enabled.

Further, the pressure sensor 74 does not have to be provided inside of the insertion part 22 in order to detect the internal pressure of the balloon 60 accurately, and the insertion part 22 can be made compact.

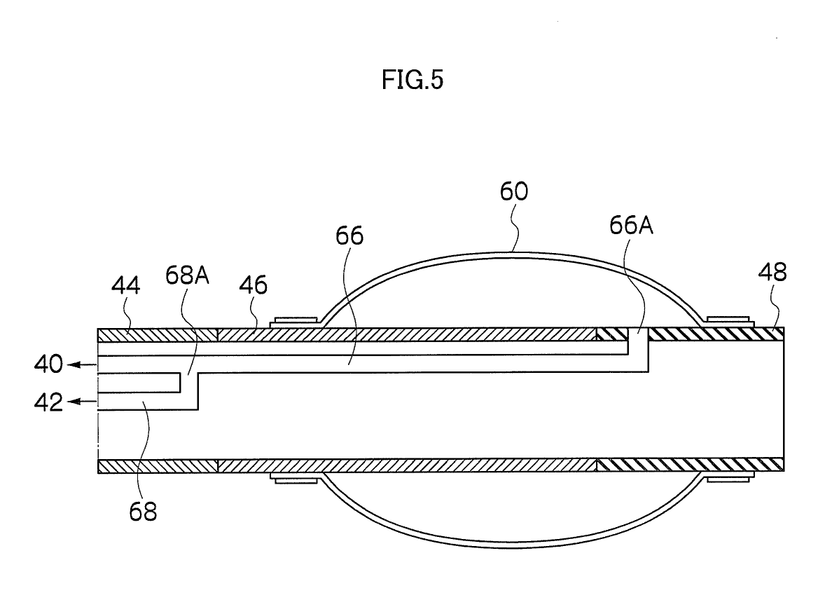

As a modified example, as shown in FIG. 5, the opening 68A which is provided at one end side of the pressure detection conduit line 68 may be connected to the supply and exhaust conduit line 66 in the vicinity of a portion where the flexible portion 44 of the insertion portion 22 is connected to the bending portion 46, in the vicinity of the balloon 60.

In the modified example in which in the vicinity of the balloon 60, the pressure detection conduit line 68 is connected to the supply and exhaust conduit line 66, the influence of the pressure loss of the fluid which occurs in the supply and exhaust conduit line 66 is small, and therefore, the internal pressure of the balloon 60 can be accurately detected.

As one example, when the length of the entire supply and exhaust conduit line 66 is about 2 m, the length of the supply and exhaust conduit line 66 which extends from the balloon 60 to the pressure sensor 74 is 2 m as it is in the prior art, whereas in this modified example, the length is only about 10 cm. Pressure loss is proportional to the length of the conduit line in which the fluid flows, and therefore, the amount which occurs as a detection error of the internal pressure of the balloon 60 is reduced to 1/20 as compared with the prior art. Even in the endoscope apparatus having the especially long bending part 46, the length of the supply and exhaust conduit line 66 which extends from the balloon 60 to the pressure sensor 74 can be made within 20 cm, and the amount which occurs as a detection error of the internal pressure of the balloon 60 is reduced to 1/10 as compared with the prior art. Therefore, the internal pressure of the balloon 60 can be accurately detected by the pressure sensor 74 without depending on the supply and exhaust amount of the fluid to and from the balloon 60.

The specifications may be adopted, in which the supply and exhaust conduit line 66 and the pressure detection conduit line 68 are formed on the outer surface of the insertion part 22, other than the specifications in which they are provided inside the insertion part 22.

From above, the internal pressure detecting device of the balloon 60 of the present embodiment has: the pressure detection conduit line 68 that is provided separately from the supply and exhaust conduit line 66 through which the fluid is supplied and exhausted to and from the balloon 60 at the insertion part 22 which is inserted into a body cavity, the pressure detection conduit line 68 communicates with the inside of the balloon 60; and the pressure sensor 74 which is connected to the pressure detection conduit line 68 and detects the internal pressure of the balloon 60. Therefore, it is possible to accurately detect the internal pressure of the balloon 60.

### Second Embodiment

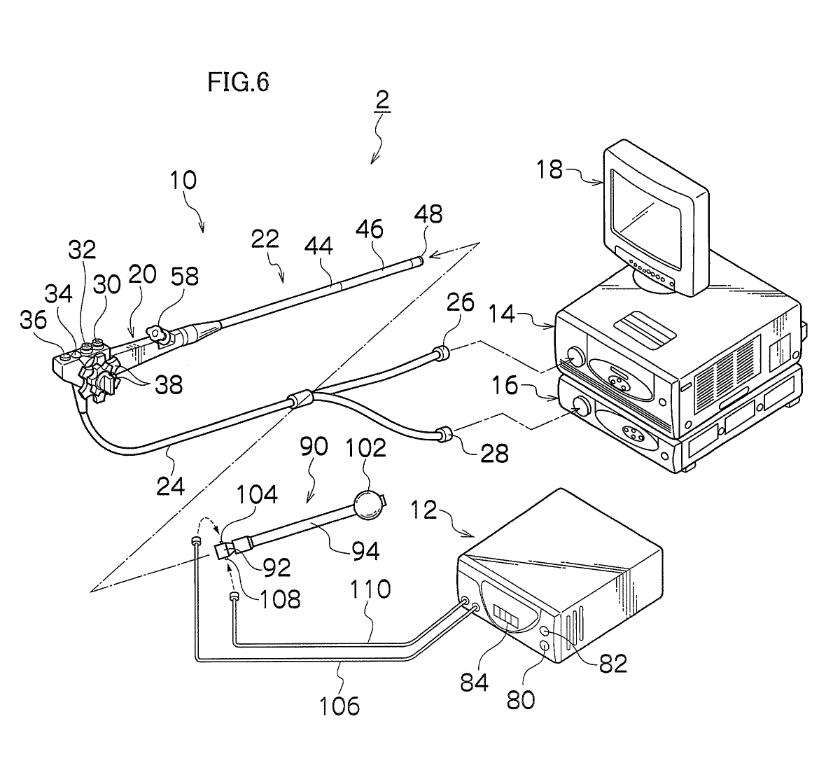

Next, FIG. 6 is a system configuration view showing one example of an endoscope apparatus 2 provided with an insertion auxiliary tool 90. The endoscope apparatus 2 of a second embodiment has the insertion auxiliary tool 90 as a main difference in the configuration from the endoscope apparatus 1 of first embodiment.

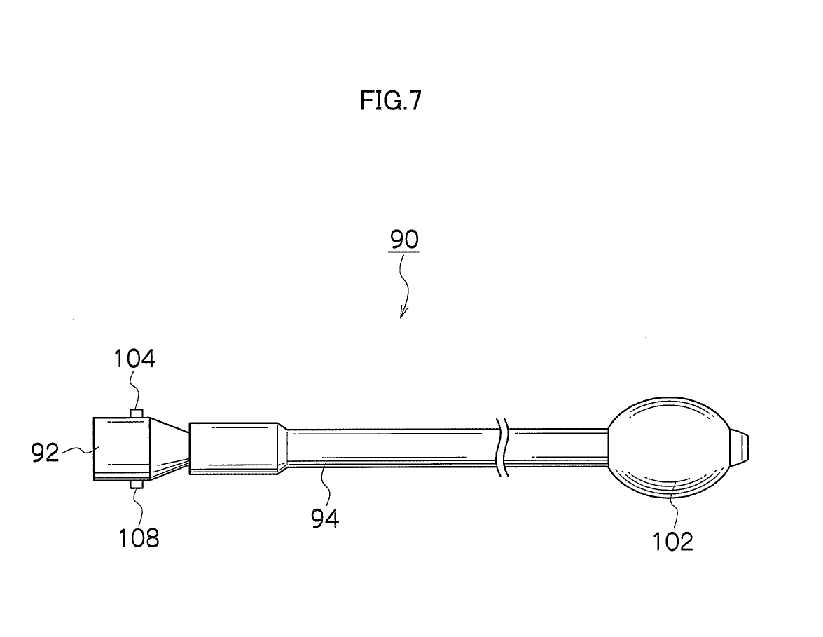

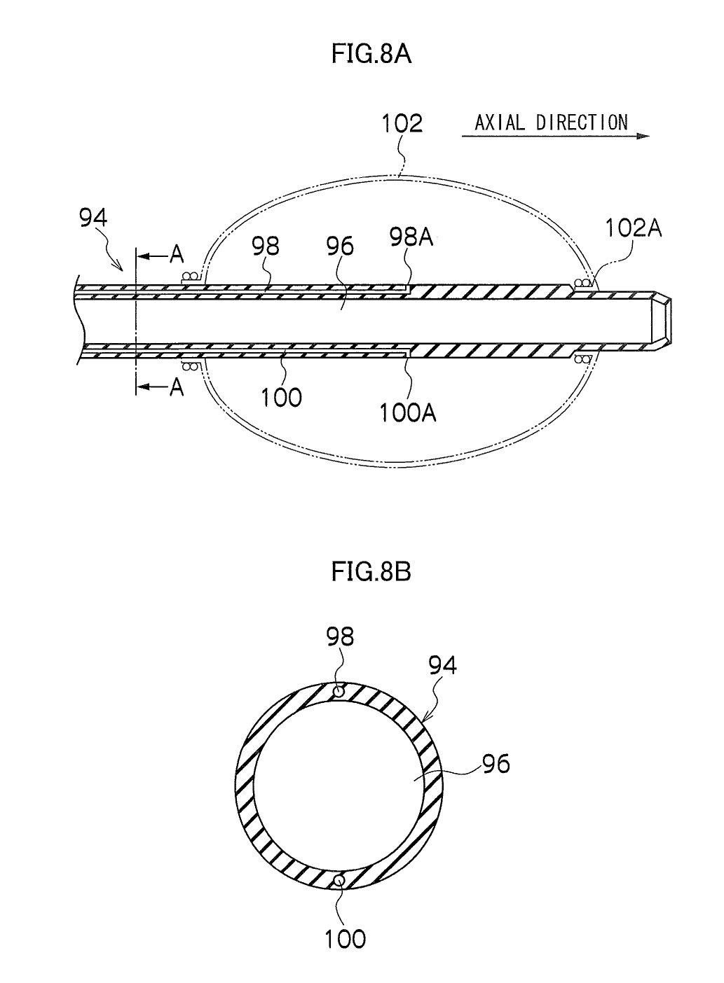

FIG. 7 is a plane view showing the insertion auxiliary tool 90, and FIGS. 8A and 8B are sectional views of a distal end portion of a tube main body 94.

As shown in FIG. 7, the insertion auxiliary tool 90 includes a grip part 92 and the tube main body 94. The grip part 92 is a part for an operator to grip, is formed into a cylindrical shape by a hard material such as plastic. A supply and exhaust connector 104 and a pressure detection connector 108 are provided at a cylindrical portion in a circumferential direction with their phases shifted from each other. The tube main body 94 is fitted onto the distal end side of the grip part 92.

The tube main body 94 is formed into a substantially cylindrical shape by a flexible material such as polyurethane. The tube main body 94 includes a balloon 102 at a distal end side opposite from the side in which the grip part 92 is fitted.

As shown in FIG. 8A, an insertion path 96, a supply and exhaust conduit line 98 and a pressure detection conduit line 100 are formed inside the tube main body 94 in an axial direction.

The insertion path 96 is a hole through which the insertion part 22 (see FIG. 6) of the endoscope 10 is inserted, a sectional shape thereof orthogonal to the axial direction is circular, and the inside diameter is formed to be a little larger than the outside diameter of the insertion part 22 (see FIG. 8B). The inner peripheral surface of the insertion path 96 is coated with a hydrophilic coat material (lubricant coat material) such as polyvinyl pyrrolidone, and a lubricant such as water is supplied to the inner peripheral surface (namely, a gap between the tube main body 94 and the insertion part 22) of the insertion path 96, whereby friction between the tube main body 94 and the insertion part 22 can be reduced.

The supply and exhaust conduit line 98 is a conduit line for supplying and exhausting the fluid (for example, air) of the balloon 102, and is formed in the axial direction within (inside) a tube wall of the insertion path 96 (see FIG. 8B). That is, the supply and exhaust conduit line 98 is formed by making a line (hole) which runs parallel to the axial direction of the insertion path 96 within the tube wall of the insertion path 96 (see FIG. 8B). The distal end side of the supply and exhaust conduit line 98 communicates with the balloon 102 through an opening 98A which is formed in an outer peripheral surface of the tube main body 94 inside the balloon 102 and is included in the inside of the balloon 102.

Meanwhile, the proximal end side of the supply and exhaust conduit line 98 communicates with a supply and exhaust connector 104 (see FIG. 7) of the grip part 92. The pump 72 (see FIG. 4) of the balloon control device 12 is connected to the supply and exhaust connector 104 through a tube 106 (see FIG. 6). Like this, the supply and exhaust conduit line 98 is connected to the pump 72. A thin tube may be connected to the proximal end side of the supply and exhaust conduit line 98, and a supply and exhaust connector 104 may be provided at the end portion of the tube.

As shown in FIG. 8A, the pressure detection conduit line 100 is a conduit line for detecting the internal pressure of the balloon 102, and is formed in the axial direction within (inside) the tube wall of the insertion path 96 similarly to the supply and exhaust conduit line 98. That is, the pressure detection conduit line 100 is formed by making a line (hole) which runs parallel to the axial direction of the insertion path 96 within the tube wall of the insertion path 96 (see FIG. 8B). The distal end side of the pressure detection conduit line 100 is configured to communicate with the balloon 102 through an opening 100A which is formed in the outer peripheral surface of the tube main body 94 and is included in the inside of the balloon 102.

Meanwhile, the proximal end side of the pressure detection conduit line 100 is configured to communicate with the pressure detection connector 108 (see FIG. 7) of the grip part 92. The pressure sensor 74 (see FIG. 4) of the balloon control device 12 is connected to the pressure detection connector 108 through the tube 110 (see FIG. 6). In this manner, the pressure detection conduit line 100 is connected to the pressure sensor 74. A thin tube may be connected to the proximal end side of the pressure detection conduit line 100, and a pressure detection connector 108 may be provided at the end portion of the tube.

As shown in FIG. 8B, the supply and exhaust conduit line 98 and the pressure detection conduit line 100 are provided at substantially symmetrical positions with respect to the center axis of the tube main body 94 with the insertion path 96 interposed therebetween. The positional relationship of the supply and exhaust conduit line 98 and the pressure detection conduit line 100 is not limited to the example of FIG. 8B, but may be any positional relationship so long as they are disposed in the positions with their phases shifted from each other in the circumferential direction of the tube main body 94.

The other configuration of the endoscope apparatus 2 of second embodiment is substantially common to the endoscope apparatus 1 of first embodiment.

Next, an example of an operation method of the endoscope apparatus 2 configured as described above will be described.

First, the insertion part 22 of the endoscope 10 is inserted through the insertion path 96 of the insertion auxiliary tool 90. Next, the insertion part 22 and the insertion auxiliary tool 90 are alternately inserted by a push method, and the balloon 102 is inflated, if needed, to fix the insertion auxiliary tool 90 to the inside of the body (for example, a large intestine). Next, the insertion auxiliary tool 90 is moved in the escaping direction (retreated) to simplify the canal shape of the inside of the body (for example, a large intestine), and thereafter, the insertion part 22 is further inserted into a deep part.

For example, the insertion part 22 is inserted from the anus of the subject, and when the distal end of the insertion part 22 passes the colon sigmoideum, the balloon 102 is inflated to fix the insertion auxiliary tool 90 to the enteric canal. Next, the insertion auxiliary tool 90 is pulled to make the colon sigmoideum substantially rectilinear. Then, the distal end of the insertion part 22 is inserted into the deep part of the enteric canal. Thereby, the insertion part 22 can be inserted into the deep part of the enteric canal.

When the balloon 102 is to be inflated or deflated, the internal pressure of the balloon 102 is detected by the pressure sensor 74, and the pump 72 is controlled.

Here, in the present embodiment, the pressure detection conduit line 100 is provided separately from the supply and exhaust conduit line 98 as shown in FIGS. 8A and 8B. The internal pressure of the balloon 102 is detected by the pressure sensor 74 via the pressure detection conduit line 100. Therefore, the internal pressure of the balloon 102 can be accurately detected by the pressure sensor 74 without being influenced by the pressure loss of the fluid which occurs in the supply and exhaust conduit line 98. Accordingly, until the internal pressure of the balloon 102 actually reaches the predetermined pressure, the pump 72 can be controlled to maximize its capacity when the fluid is supplied into the balloon 102, or exhausted from the inside of the balloon 102, and safe and quick supply and discharge are enabled.

Further, the pressure sensor 74 does not have to be provided at the tube main body 94 in order to detect the internal pressure of the balloon 102 accurately, and the insertion auxiliary tool 90 can be made compact.

As a modified example, one end side of the pressure detection conduit line 100 may be connected to the supply and exhaust conduit line 98 in the vicinity of the balloon 102 instead of being directly opened to the balloon 102.

In this modified example, also, the length of the supply and exhaust conduit line 98 which extends from the balloon 102 to the pressure sensor 74 becomes short, and the influence of the pressure loss of the fluid which occurs in the supply and exhaust conduit line 98 becomes small. Therefore, the internal pressure of the balloon 102 can be accurately detected. Therefore, the internal pressure of the balloon 102 can be accurately detected by the pressure sensor 74 without being influenced by the pressure loss of the fluid which occurs in the supply and exhaust conduit line 98.

The specifications may be adopted, in which the supply and exhaust conduit line 98 and the pressure detection conduit line 100 are formed on the outer surface of the tube main body 94, other than the specifications in which they are provided inside the tube main body 94.

From above, the internal pressure detecting device of the balloon 102 of the present embodiment has the pressure detection conduit line 100 that is provided separately from the supply and exhaust conduit line 98 through which the fluid inside the balloon 102 provided at the insertion auxiliary tool 90 which is inserted into a body cavity is supplied and exhausted, and is caused to communicate with the inside of the balloon 102, and the pressure sensor 74 which is connected to the pressure detection conduit line 100 and detects the internal pressure of the balloon 102. Therefore, it is possible to accurately detect the internal pressure of the balloon 102.

### Third Embodiment

Next, FIG. 9 is a system configuration view showing one example of an endoscope apparatus 3 provided with an insertion guide tool 120.

The endoscope apparatus 3 of a third embodiment has an insertion guide tool 120 as a main difference in the configuration from the endoscope apparatus 1 of the first embodiment. As shown in FIG. 9, the insertion guide tool 120 is inserted from the forceps insertion part 58, and is led out from the forceps port 56 (see FIG. 2) of the distal end part 48.

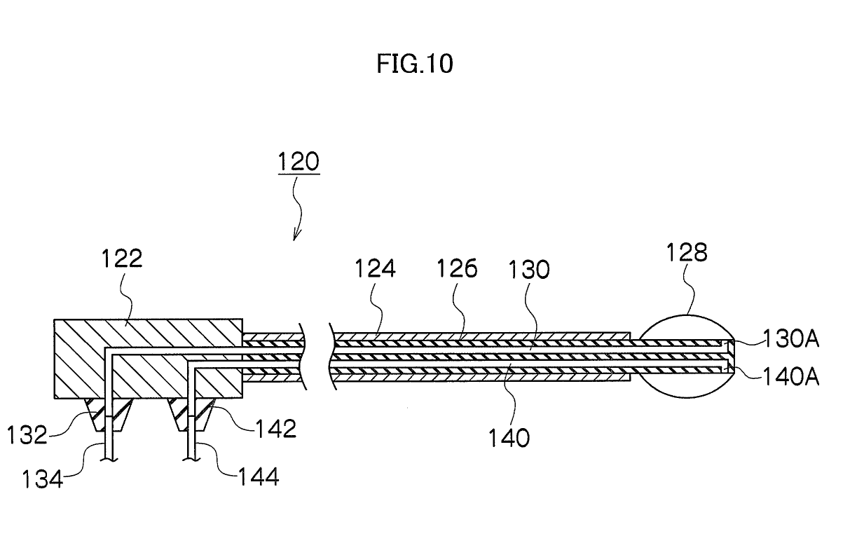

FIG. 10 is a sectional view of a side surface of the insertion guide tool 120.

As shown in FIG. 10, the insertion guide tool 120 includes a hard grip part 122, a cylindrical outer sheath member 124 connected to the grip part 122, and a flexible member (corresponding to a linear member) 126 inserted into the outer sheath member 124.

The flexible member 126 is formed into a linear shape, has sufficient flexibility, and its proximal end is fixed to the grip part 122. A balloon 128 is fitted on an outer peripheral surface of a distal end of the flexible member 126. The balloon 128 is formed by an elastic body such as rubber, is inflated into a substantially semispherical shape by supplying a fluid into an inside, and is stuck onto an outer peripheral surface of the flexible member 126 by sucking the fluid from the inside. As a fluid for use, air, inert gas, water and the like can be properly selected.

Inside the flexible member 126, a supply and exhaust conduit line 130 which communicates with a connector 132 formed in the grip part 122, and supplies and exhausts the fluid in the balloon 128 is formed. A distal end side of the supply and exhaust conduit line 130 communicates with the balloon 128 through an opening 130A which is formed in an outer peripheral surface of the flexible member 126 inside the balloon 128 and is included inside the balloon 128.

Meanwhile, a proximal end side of the supply and exhaust conduit line 130 is connected to the pump 72 (see FIG. 4) of the balloon control device 12 via a connector 132 and a tube 134. In this manner, the supply and exhaust conduit line 130 is connected to the pump 72.

Further, inside the flexible member 126, a pressure detection conduit line 140, which communicates with a connector 142 formed at the aforementioned grip part 122, is formed. A distal end side of the pressure detection conduit line 140 is caused to communicate with the balloon 128 through an opening 140A which is formed in the outer peripheral surface of the flexible member 126, and is included inside the balloon 128.

Meanwhile, a proximal end side of the pressure detection conduit line 140 is connected to the pressure sensor 74 (see FIG. 4) of the balloon control device 12 via the connector 142 and the tube 144. In this manner, the pressure detection conduit line 140 is connected to the pressure sensor 74.

The other configuration of the endoscope apparatus 3 of third embodiment is substantially common to the endoscope apparatus 1 of first embodiment.

Next, an example of an operation method of the endoscope apparatus 3 configured as described above will be described.

First, the insertion part 22 of the endoscope 10 is inserted into an enteric canal (for example, a descending limb of duodenum) with the balloon 60 deflated. Next, the balloon 60 is inflated and locked to the enteric canal, and the insertion part 22 is fixed to the enteric canal.

Next, with the balloon 128 deflated, the insertion guide tool 120 is inserted from the forceps insertion part 58 of the hand operation part 20, the distal end of the insertion guide tool 120 is led out from the insertion part 22, and is inserted into the deep part of the enteric canal.

Next, the balloon 128 is inflated and locked to the enteric canal, and the insertion part 22 is fixed to the enteric canal. Next, after the fluid is sucked from the balloon 60 and the balloon 60 is deflated, the insertion part 22 is pushed in, and is inserted along the insertion guide tool 120. Next, the balloon 60 at the distal end of the insertion part 22 is inflated at the position in the vicinity of the proximal end side of the balloon 128. Thereby, the enteric canal is gripped by the balloon 60.

Next, after the insertion part 22 is pulled in, and the balloon 128 is deflated by sucking the fluid from the balloon 128, the insertion guide tool 120 is inserted into a deep part of the enteric canal. Thereafter, the above insertion operation is repeated in sequence. Thereby, the insertion part 22 can be inserted into a deep part of the enteric canal.

When the balloon 128 is inflated or deflated, the internal pressure of the balloon 128 is detected by the pressure sensor 74, and the pump 72 is controlled.

Here, in the present embodiment, as shown in FIG. 10, the pressure detection conduit line 140 is provided separately from the supply and exhaust conduit line 130. The internal pressure of the balloon 128 is detected by the pressure sensor 74 through the pressure detection conduit line 140. Therefore, the internal pressure of the balloon 128 can be accurately detected by the pressure sensor 74 without being influenced by the pressure loss of the fluid which occurs in the supply and exhaust conduit line 130. Accordingly, until the internal pressure of the balloon 128 reaches the predetermined pressure, the pump 72 can be controlled to maximize its capacity when the fluid can be supplied into the balloon 128 or is exhausted from the inside of the balloon 128, thereby safe and quick supply and exhaust are enabled.

Further, the pressure sensor 74 does not have to be provided at the insertion guide tool 120 in order to detect the internal pressure of the balloon 128 accurately, and the insertion guide tool 120 can be made compact.

As a modified example, one end side of the pressure detection conduit line 140 may be connected to the supply and exhaust conduit line 130 in the vicinity of the balloon 128 without being directly opened to the balloon 128.

In this modified example, the length of the supply and exhaust conduit line 130 which extends from the balloon 128 to the pressure sensor 74 becomes short, and the influence of the pressure loss of the fluid which occurs in the supply and exhaust conduit line 130 becomes small. Therefore, the internal pressure of the balloon 128 can be accurately detected. Therefore, the internal pressure of the balloon 128 can be accurately detected by the pressure sensor 74 without being influenced by the pressure loss of the fluid which occurs in the supply and exhaust pipe line 130.

The specifications may be adopted, in which the supply and exhaust conduit line 130 and the pressure detection conduit line 140 are formed on the outer surface of the outer sheath member 124, other than the specifications in which they are provided inside the flexible member 126.

As above, the internal pressure detecting device of the balloon 128 of the present embodiment has: the pressure detection conduit line 140 which is provided separately from the supply and exhaust conduit line 130 through which the fluid inside the balloon 128 provided at the insertion guide tool 120 which is inserted into the body cavity is supplied and exhausted, and is caused to communicate with the inside of the balloon 128; and the pressure sensor 74 which is connected to the pressure detection conduit line 140 and detects the internal pressure of the balloon 128. Therefore, it is possible to accurately detect the internal pressure of the balloon 128.

Further, as above, the endoscope apparatuses 1 to 3 of the embodiments 1 to 3 have the controllers 70 which control the internal pressures of the balloons 60, 102 and 128 by controlling the pumps 72 based on the detection results of the pressure sensors 74. Therefore, until the internal pressures of the balloons 60, 102 and 128 reach the predetermined pressures, the pump 72 can be controlled to maximize its capacity. Thereby, stable and quick supply and exhaust of the fluid are enabled.

### Other Embodiments

As the other embodiments, the following embodiments are also conceivable.

The above described embodiments 1 to 3 may be properly combined.

For example, in the endoscope apparatus 2 of second embodiment, the balloon 60 of first embodiment may be further provided at the insertion part 22. In addition, the insertion auxiliary tool 90 is provided with the pressure detection conduit line 100, the insertion part 22 is also provided with the pressure detection conduit line 68, and two pressure sensors which respectively connect to the pressure detection conduit line 100 and the pressure detection conduit line 68 are provided in the balloon control device 12. Thereby, the internal pressures of the balloon 102 and the balloon 60 may be made detectable by the pressure sensors through the respective pressure detection conduit line 100 and pressure detection conduit line 68.

Similarly, the combination of first embodiment and third embodiment, the combination of second embodiment and third embodiment, and the combination of first embodiment and second embodiment and third embodiment are also conceivable.

Further, in the above described embodiments, the pressure sensors 74 are provided in the balloon control devices 12 via the pressure detection conduit lines 68, 100 and 140. Other than them, embodiments are also conceivable, in which the pressure sensors are directly provided at the balloons 60, 102 and 128. In these cases, as the pressure sensors, piezoelectric type pressure sensors, strain gauges and the like can be used.

According to these embodiments, the internal pressures of the balloons 60, 102 and 128 can be accurately detected without being influenced by the pressure losses which occur in the supply and exhaust conduit lines 66, 98 and 130. Therefore, until the internal pressures of the balloons 60, 102 and 128 reach predetermined pressures, the pump 72 is controlled to maximize its capacity, and safe and quick supply or exhaust is enabled.

Further, an embodiment is also conceivable, in which a pressure sensor is provided in the vicinity of the balloon 60 in the supply and exhaust conduit line 66 in the flexible part 44 of the insertion part 22 of the endoscope 10, that is, in the vicinity of the connecting part to the bending part 46. Further, an embodiment is also conceivable, in which a pressure sensor is provided in the vicinity of the balloon 102 in the supply and exhaust conduit line 98 of the insertion auxiliary tool 90. Further, an embodiment is also conceivable, in which a pressure sensor is provided in the vicinity of the balloon 128 in the supply and exhaust conduit line 130 of the insertion guide tool 120.

According to these embodiments, the lengths of the supply and exhaust conduit lines 66, 98 and 130 which extends from the balloons 60, 102 and 128 to the pressure sensors become short, and the influence of the pressure losses which occur in the supply and exhaust conduit lines 66, 98 and 130 become small. Therefore, the internal pressures of the balloons 60, 102 and 128 can be accurately detected.

Further, especially according to the embodiment in which the pressure sensor is provided in the vicinity of the connecting part to the bending part 46 in the supply and exhaust conduit line 66 in the flexible part 44 of the insertion part 22 of the endoscope 10, the pressure sensor is provided in a region other than regions where the components are mounted with high density, such as the bending part 46 and the distal end part 48. Therefore, the endoscope 10 can be made compact.

The internal pressure detecting device for the inflating and deflating member and the endoscope apparatus of the presently disclosed subject matter will be described in detail above, but the presently disclosed subject matter is not limited to the above examples, various improvements and modifications may be made in the range without departing from the gist of the presently disclosed subject matter as a matter of course.

What is claimed is:

1. An internal pressure detecting device for an inflating and deflating member, comprising:
a pressure detection conduit line which is provided separately from a supply and exhaust conduit line through which a fluid is supplied to or exhausted from an inside of an inflating and deflating member provided at an intracavital insertion tool, and which communicates with the inside of the inflating and deflating member; and
a pressure detecting device which is connected to the pressure detection conduit line and detects an internal pressure of the inflating and deflating member.

2. The internal pressure detecting device for an inflating and deflating member according to claim 1,
wherein an opening of the pressure detection conduit line is included in the inside of the inflating and deflating member.

3. The internal pressure detecting device for an inflating and deflating member according to claim 1,
wherein an opening of the pressure detection conduit line is included in a vicinity of the inflating and deflating member in the supply and exhaust conduit line.

4. The internal pressure detecting device for an inflating and deflating member according to claim 1,
wherein the pressure detecting device is disposed outside the intracavital insertion tool.

5. The internal pressure detecting device for an inflating and deflating member according to claim 1,
wherein the pressure detection conduit line is provided inside the intracavital insertion tool.

6. The internal pressure detecting device for an inflating and deflating member according to claim 1,
wherein the intracavital insertion tool is an insertion part of an endoscope.

7. The internal pressure detecting device for an inflating and deflating member according to claim 1,
wherein the intracavital insertion tool is an endoscope auxiliary tool through which an insertion part of an endoscope is inserted.

8. The internal pressure detecting device for an inflating and deflating member according to claim 1,
wherein the intracavital insertion tool is an endoscope treatment tool which is led out from a forceps port of an endoscope.

9. The internal pressure detecting device for an inflating and deflating member according to claim 3,
wherein the intracavital insertion tool is an insertion part of an endoscope,
the insertion part includes, in sequence from a proximal end side:
a flexible part having flexibility;
a bending part which is connected to a proximal end side of the flexible part and is bendable; and
a distal end part which can be directed to a desired direction by bending the bending part, and
the vicinity of the inflating and deflating member is a vicinity of a part where the bending part is connected to the flexible part.

10. An endoscope apparatus, comprising:
an intracavital insertion tool which is inserted into a body cavity;
an inflating and deflating member provided at the intracavital insertion tool;
a supply and exhaust conduit line through which a fluid is supplied to or exhausted from an inside of the inflating and deflating member;
a supply and exhaust device which is connected to the supply and exhaust conduit line and supplies or exhausts the fluid to or from the inside of the inflating and deflating member;
a pressure detection conduit line which is provided separately from the supply and exhaust conduit line and communicates with the inside of the inflating and deflating member;
a pressure detecting device which is connected to the pressure detection conduit line and detects an internal pressure of the inflating and deflating member; and
an internal pressure control section which controls the internal pressure of the inflating and deflating member by controlling the supply and exhaust device based on a detection result of the internal pressure detecting device.
